(19) United States
(12) Patent Application Publication
(10) Pub. No.: US 2023/0144192 A1
(43) Pub. Date: May 11, 2023

(54) TREATMENT OF OSTEOGENESIS IMPERFECTA

(71) Applicant: Genzyme Corporation, Cambridge, MA (US)

(72) Inventors: Panteleimon D. MAVROUDIS, Cambridge, MA (US); Nikhil PILLAI, Andover, MA (US); Qingping WANG, Acton, MA (US)

(73) Assignee: Genzyme Corporation, Cambridge, MA (US)

(21) Appl. No.: 18/051,468

(22) Filed: Oct. 31, 2022

Related U.S. Application Data
(60) Provisional application No. 63/274,503, filed on Nov. 1, 2021.

(30) Foreign Application Priority Data
Oct. 13, 2022 (EP) 22315238.0

(51) Int. Cl. C07K 16/22; A61K 47/64; A61K 39/395; A61K 31/663; A61K 31/675; A61K 38/23; A61K 38/29; A61K 45/06; A61P 19/08

(52) U.S. Cl. CPC C07K 16/22; A61K 47/6455; A61K 39/3955; A61K 31/663; A61K 31/675; A61K 38/23; A61K 38/29; A61K 45/06; A61P 19/08; A61K 2039/545

(57) ABSTRACT

The present disclosure provides methods for treating and improving b1osteogenesis imperfecta (OI) in a subject by administering to the subject a therapeutically effective amount of an agent that binds and neutralizes transforming growth factor beta (TGF-β).

### CROSS REFERENCE TO RELATED APPLICATION

This application claims priority from U.S. Provisional Application 63/274,503, filed on Nov. 1, 2021, and European Application No. 22315238.0, filed on Oct. 13, 2022. The disclosures of the two priority applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 26, 2022, is named 022548US028.XML and is 20,162 bytes bytes in size.

### BACKGROUND OF THE INVENTION

Osteogenesis Imperfecta (OI) is a genetically and phenotypically heterogeneous Mendelian disorder of connective disorder that has an estimated prevalence of 1 in 10,000-20,000 births. The skeletal manifestations of OI include low bone mass, bone fragility, recurrent fractures, scoliosis, and bone deformities. The extraskeletal manifestations include decreased muscle mass, muscle weakness, dentinogenesis imperfecta, hearing loss, and pulmonary disease (Marini, Nat Rev Dis Primers (2017)3:17052; Marom et al., Am J Med Genet C Semin Med Genet. (2016) 172 (4):367-83; Patel et al., Clin Gen. (2015) 87 (2):133-40; Rossi et al., Curr Opin Pediatr. (2019) 31 (6):708-15; Tam et al., Clin Gen. (2018) 94 (6):502-11; DiMeglio et al. J Bone Miner Res. (2006) 21:132-40; Gatti et al., J Bone Miner Res. (2005) 20 (5):758-63); Gatti et al., Calcified Tissue Int (2013) 93 (5):448-52). The management of individuals with OI typically involves a multidisciplinary approach. The mainstay therapy for OI bone fragility involves repurposing of medications that are used to treat osteoporosis (Adami et al., J Bone Miner Res. (2003) 18 (1):126-30; Bishop et al., Ear Hum Dev. (2010) 86 (11):743-6; Chevrel et al., J Bone Miner Res. (2006) 21 (2):300-6; Glorieux et al., NEJM (1998) 339 (14):947-52; Rauch et al., J Bone Miner Res. (2009) 24 (7):1282-9; Rauch et al., J Bone Miner Res. (2003) 18 (4):610-4; Orwoll et al., J Clin Invest (2014) 124 (2):491-8; Hoyer-Kuhn et al., J Musculoskelet Neuronal Interact. (2016) 16 (1):24-32; Anissipour et al., J Bone Joint Surg Am. (2014) 96 (3):237-43).

Despite significant progress, most pharmacological interventions in OI have not provided the disease-modifying effects observed in other bone diseases such as osteoporosis and are less effective in certain types of OI. Due to the complexity of the underlying biochemical processes involved in OI and bone remodeling, translation of the Pharmacokinetic/Pharmacodynamic (PK/PD) relationship in the clinic is challenging. Bisphosphonates (BPN), a class of antiresorptive medications that decrease bone resorption, is currently the standard of care, especially in pediatric OI. In children, BPN has been shown to have beneficial effects on areal and volumetric bone mineral density (aBMD and vBMD), progression of scoliosis, quality-of-life, and in some studies, fracture incidence (Bishop et al., Lancet (2013) 382 (9902):1424-32; Rauch et al., 2003, supra; Bains et al., JBMR Plus (2019) 3 (5):e10118; Rauch et al., Bone (2007) 40 (2):274-80). However, given the heterogeneity of OI and the variability in the clinical study designs, the effects of BPN have been inconsistent. In adults, the benefits and the consequences of long-term treatment with bisphosphonates are less certain (Adami et al., ibid; Shi et al., Am J Ther. (2016) 23 (3):e894-904). Additionally, in a randomized trial involving adults with OI, treatment with an anabolic agent, teriparatide, led to increase in aBMD and vBMD in individuals with the mild form (OI type I) but not in the moderate-to-severe forms of the disorder (0I types III and IV). Furthermore, none of these repurposed therapies address specific pathogenetic mechanism in OI, and hence, they have no effect on extraskeletal manifestations.

Therefore, there remains a significant unmet need for effective therapy targeting various forms of OI.

### SUMMARY OF THE INVENTION

The present disclosure provides a method for treating osteogenesis imperfecta (OI) in a human subject in need thereof, comprising administering to the subject a therapeutically effective amount of an anti-TGF-β antibody, wherein the antibody comprises heavy chain complementarity-determining regions (CDRs) 1-3 comprising SEQ ID NOs:4-6, respectively, light chain CDR1-3 comprising SEQ ID NOs:7-9, respectively, wherein the antibody comprises a human IgG₄ constant region having a proline at position 228 (Eu numbering), and wherein the therapeutic effective amount is 1 to 8 mg/kg, optionally 2, 2.5, or 5 mg/kg, administered bi-annually (e.g., every six months or Q6M), or 0.1 to 1 mg/kg, optionally 0.35, 0.4, or 0.5 mg/kg, administered every 3 months (Q3M).

In some embodiments, the antibody herein comprises a heavy chain variable domain comprising SEQ ID NO:10 and a light chain variable domain comprising SEQ ID NO:11. In further embodiments, the antibody comprises a human IgG₄ constant region and/or a human κ light chain constant region. In certain embodiments, the antibody comprises or consists of a heavy chain comprising SEQ ID NO:3 and a light chain comprising SEQ ID NO:2.

In some embodiments, the antibody comprises a bone-targeting moiety, optionally wherein the bone-targeting moiety is a poly-arginine peptide (e.g., SEQ ID NO:14). In further embodiments, the antibody comprises one or more poly-arginine peptides. In certain embodiments, the antibody is fused to a poly-arginine peptide at the N-terminus, or the C-terminus, or both termini, of the heavy chain, and/or at the C-terminus of the light chain, of the antibody.

In some embodiments, the OI to be treated herein is moderate-to-severe OI or type IV OI. In some embodiments, the OI is type I, II, or III.

In some embodiments, the human subject is an adult patient (≥18 years of age), or a pediatric patient (<18 years of age). In some embodiments, the human subject has a mutation in a COL1A1 or COL1A2 gene, optionally wherein the mutation is a glycine substitution mutation in the COL1A1 or COL1A2 gene or a valine deletion in the COL1A2 gene.

In some embodiments, the treatment herein improves a bone parameter selected from the group consisting of bone mineral density (BMD), bone volume density (BV/TV), total bone surface (BS), bone surface density (BS/BV), trabecular number (Tb.N), trabecular thickness (Tb.Th), trabecular spacing (Tb.Sp), and total volume (Dens TV).

In some embodiments, the treatment herein decreases bone turnover and/or osteocyte density, optionally wherein the decreased bone turnover is indicated by a decrease in serum CTX or an increase in serum osteocalcin (OCN).

In some embodiments, the antibody is administered at 2 mg/kg bi-annually or at 0.4 mg/kg Q3M, optionally wherein the administration leads to an increase of BMD in the subject by about 5%. In some embodiments, the antibody is administered at 5 mg/kg bi-annually or at 0.5 mg/kg Q3M, optionally wherein the administration leads to an increase of BV in the subject by about 5%. In some embodiments, the antibody is administered at 2.5 mg/kg bi-annually or at 0.35 mg/kg Q3M, optionally wherein the administration leads to a decrease of the TGF-β level in the subject to the homeostatic value.

In some embodiments, the antibody is administered by intravenous infusion. In some embodiments, the treatment herein includes another therapeutic agent, such as a bisphosphonate, parathyroid hormone, calcitonin, teriparatide, or an anti-sclerostin agent. In further embodiments, the bisphosphonate is selected from alendronate, pamidronate, zoledronate, and risedronate.

Also provided herein are an anti-TGF-β antibody for use in treating osteogenesis imperfecta in the present treatment method; use of an anti-TGF-β antibody in the manufacture of a medicament for treating osteogenesis imperfecta in the method; and an article of manufacture or kit, comprising an anti-TGF-β antibody for use in treating osteogenesis imperfecta in the method.

Also provided herein are an anti-TGF-β antibody or an antigen-binding fragment thereof for use in treating osteogenesis imperfecta in the treatment method herein, and use of an anti-TGF-β antibody or an antigen-binding fragment thereof in the manufacture of a medicament for treating osteogenesis imperfecta in the treatment method herein.

Also provided is an article of manufacture (e.g., a kit), comprising an anti-TGF-β antibody or an antigen-binding fragment thereof for use in treating osteogenesis imperfecta in the treatment method herein.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIGS. 1A-C are graphs illustrating a multi-model approach to evaluate the concentration response relationship of Ab1 (GC2008) on bone mass density (BMD), bone strength, and TGF-β dynamics in bone of OI patients. FIG. 1A illustrates PK/PD modeling based on clinical data using fresolimumab (GC1008), a fully human anti-TGF-β antibody. FIG. 1B illustrates PK/PD modeling based on pre-clinical data using 1D11, a mouse anti-TGF-β antibody (U.S. Pat. No. 5,571,714; ATCC Deposit #HB9849; available at, e.g., Thermo Fisher, Cat. #MA5-23795). FIG. 1C illustrates physiological based pharmacokinetic modeling (PBPK) based on physiochemical (PC) properties of Ab1, another fully human anti-TGF-β antibody.

FIG. 2 is a pair of graphs showing the use of PK data of fresolimumab (1 mg/kg or 4 mg/kg intravenous (“IV”) administration) in the serum of focal segmental glomerulosclerosis (FSGS) patients in informing PK/BMD response of fresolimumab in OI patients.

FIG. 3 is a pair of graphs showing the use of Ab1 PK data in predicting PK/BMD response of Ab1 in OI patients.

FIG. 4 is a panel of graphs showing the use of mouse 1D11 PK data in predicting PK/BV response of Ab1 in OI patients. Graph A shows concentration vs time (left-axis), and bone volume fraction (right-axis) for 5 mg/kg administration 3 times per week in mice. Graph B shows concentration vs time (left-axis), and bone volume fraction (right-axis) for 5 mg/kg administration weekly in mice. Graph C shows concentration vs time (left-axis), and bone volume fraction (right-axis) for 5 mg/kg administration every two weeks in mice. Graph D shows concentration vs time (left-axis), and bone volume fraction (right-axis) for 5 mg/kg administration every four weeks in mice. Graph E shows concentration vs time (left-axis), and bone volume fraction (right-axis) for 0.5 mg/kg administration every three months in humans. Graph F shows concentration vs time (left-axis), and bone volume fraction (right-axis) for 5 mg/kg administration every six months in humans. Symbols are average bone volume fraction data, and error bars depict their standard deviation.

FIG. 5 is a panel of graphs showing the use of physiochemical properties of Ab1 in modeling a physiologically-based PK (PBPK) response of Ab1 in OI patients. Graph A shows concentration of Ab1 for 0.05, 0.25, 1 and 3 mg/kg single IV administration. Symbols are individual subject data, and lines depict predictions of PBPK model. Graph B shows comparison of plasma (solid line) and bone (dotted line) PK for a single IV administration of 0.05 mg/kg Abl. Graph C shows plasma PK prediction of 0.35 mg/kg administration every 3 months and 2.5 mg/kg administration every six months. Graph D shows TGFβ target dynamics in bone, after 0.35 mg/kg administration every 3 months, or 2.5 mg/kg administration every 6 months of Ab1.

FIG. 6 is a panel of graphs showing Ab1 population PK evaluation plots. Graph A shows the observed Ab1 concentration versus individual predictions. Graph B shows the observed Ab1 concentration versus population prediction. Graph C shows the normalized prediction distribution error versus Ab1 population prediction. Graph D shows the normalized prediction distribution error versus time. Straight line indicates identity (y=x) line and curved line is the spline interpolation.

FIG. 7 is a graph showing the 1D11 PK response after 5 mg/kg IP administration in OI mice. Circles represent OI mice data and solid line one-compartment model simulation.

### DETAILED DESCRIPTION OF THE INVENTION

The present disclosure provides a method of treating OI in a human patient by administering a monoclonal antibody that binds and neutralizes all isoforms of human TGF-β. The method is developed based on a multiple model-based approach that relies on pre-clinical and clinical PK and PD data to inform the concentration response relationship of anti-TGF-β antibody Ab1 and its impact on bone mineral density (BMD), bone strength, and TGF-β -expression levels in OI patients.

### I. Osteogenesis Imperfecta

OI encompasses a group of congenital bone disorders characterized by deficiencies in one or more proteins involved in bone matrix deposition or homeostasis. There are over 19 types of OI that are defined by their specific gene mutation, the resulting protein deficiency, and phenotype of the affected individual. The classification includes findings on X-rays and other imaging tests. The main OI types are as follows (information from a website of The John Hopkins University).

Type I is the mildest and most common type. About 50% of all affected children have this type. There are few fractures and deformities

Type II is the most severe type. A baby has very short arms and legs, a small chest, and soft skull. He or she may be born with fractured bones and may also have a low birth weight and lungs that are not well developed. A baby with type II OI usually dies within weeks of birth.

Type III is the most severe type in babies who do not die as newborns. At birth, a baby may have slightly shorter arms and legs than normal and arm, leg, and rib fractures. A baby may also have a larger than normal head, a triangle-shaped face, a deformed chest and spine, and breathing and swallowing problems.

Type IV is an OI type where symptoms are between mild and severe. A baby with type IV may be diagnosed at birth. He or she may not have any fractures until crawling or walking. The bones of the arms and legs may not be straight. He or she may not grow normally.

Type V is similar to type IV. Symptoms may be medium to severe. It is common to have enlarged thickened areas (hypertrophic calluses) in the areas where large bones are fractured.

Type VI is very rare. Symptoms are medium and similar to type IV.

Type VII may be like type IV or type II. It is common to have shorter than normal height. It is also common to have shorter than normal upper arm and thighbones.

Type VIII is similar to types II and III. The patient has very soft bones and severe growth problems.

Although phenotypes vary among OI types, common symptoms include incomplete ossification of bones and teeth, reduced bone mass, brittle bones, and pathologic fractures. Specific symptoms include easily broken bones, bone deformities (such as bowing of the legs), discoloration of the white of the eye (sclera), a barrel-shaped chest, a curved spine, a triangle-shaped face, loose joints, muscle weakness, skin that easily bruises, hearing loss in early adulthood, and/or soft, discolored teeth. Complications of OI include respiratory infections (e.g., pneumonia), heart problems (e.g., poor heart valve function), kidney stones, joint problems, hearing loss, and abnormal eye conditions (including vision loss). OI may be diagnosed or monitored by X-rays, lab tests (e.g., blood test and genetic testing), dual energy X-ray absorptiometry scan (DXA or DEXA scan), and bone biopsy.

While multiple pathogenic genetic mutations can cause the various subtypes of OI, more than 90% are caused by pathogenic variants in the COL1A1 gene (which encodes collagen type I alpha 1 chain) or the COL2A1 gene (which encodes collagen type II alpha 1 chain), or genes encoding proteins that post-translationally modify type I collagen (CRTAP, PPIB and LEPRE1) (Patel et al., ibid; Lim et al., Bone (2017)102:40-49).

In some embodiments, the OI in the patient is caused by a mutation (e.g., a glycine substitution) in COL1A1 or COL1A2 or by biallelic pathogenic variants in CRTAP, PPIB, or LEPRE1.

### II. Anti-TGF-β Antibodies

TGF-β s are multifunctional cytokines that are involved in cell proliferation and differentiation, embryonic development, extracellular matrix formation, bone development, wound healing, hematopoiesis, and immune and inflammatory responses. Secreted TGF-β protein is cleaved into a latency-associated peptide (LAP) and a mature TGF-β peptide, and is found in latent and active forms. The mature TGF-β peptide forms both homodimers and heterodimers with other TGF-β family members.

There are three human (h) TGF-β isoforms: TGF-β, TGF-β, and TGF-β (UniProt Accession Nos. P01137, P08112, and P10600, respectively). TGF-β 1 differs from TGF-β 2 by 27, and from TGF-β 3 by 22, mainly conservative, amino acids. Human TGF-βs are very similar to mouse TGF-βs: human TGF-β 1 has only one amino acid difference from mouse TGF-β 1; human TGF-β 2 has only three amino acid differences from mouse TGF-β 2; and human TGF-β 3 is identical to mouse TGF-β 3.

Binding of a TGF-β protein to a homodimeric or heterodimeric TGF-β transmembrane receptor complex activates the canonical TGF-β signaling pathway mediated by intracellular SMAD proteins. Deregulation of TGF-β s leads to pathological processes that, in humans, have been implicated in numerous conditions, such as birth defects, cancer, chronic inflammatory, autoimmune diseases, and fibrotic diseases (see, e.g., Border et al., Curr Opin Nephrol Hypertens. (1994) 3 (4):446-52; Border et al., Kidney Int Suppl. (1995) 49:S59-61).

For the present OI treatment methods, the anti-TGF-β antibody may be a pan-specific antibody, i.e., an antibody that binds and neutralizes all three isoforms of TGF-β with high affinity. In some embodiments, the antibody is fresolimumab. Fresolimumab is a recombinant human antibody. Its heavy chain is shown below:

(SEQ ID NO: 1)

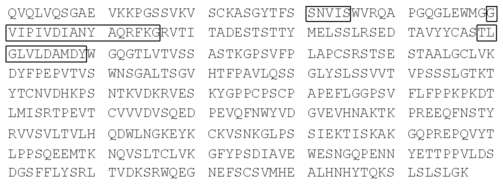

In the above sequence, positions 1-120 is the heavy chain variable domain (V_{H}), and the heavy chain CDRs (“HCDRs”; according to Kabat definition) are boxed. This heavy chain comprises a human IgG₄ constant region.

The light chain of fresolimumab is shown below:

(SEQ ID NO: 2)
ETVLTQSPGT LSLSPGERAT LSCRASQSLG SSYLAWYQQK
PGQAPRLLIY GASSRAPGIP DRFSGSGSGT DFTLTISRLE
PEDFAVYYCQ QYADSPITFG QGTRLEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC

In the above sequence, positions 1-108 is the light chain variable domain (V_{L}), and the light chain CDRs (“LCDRs”; according to Kabat definition) are underlined. This light chain comprises a human C_{κ} constant region.

In some embodiments, the anti-TGF-β antibody herein is Ab1, a variant of fresolimumab. The heavy chain of Ab1 differs from that of fresolimumab in only a residue in the IgG₄ hinge region. The residue is S228 (Eu numbering), where Ab1 has a proline at that position, i.e., having a S228P substitution relative to fresolimumab. Ab1 and fresolimumab have the same light chain. The heavy chain of Ab1 is shown below:

(SEQ ID NO: 3)

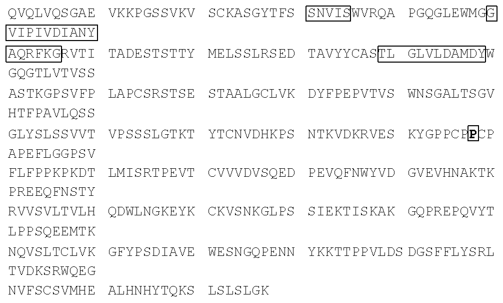

In the above sequence, the HCDRs are boxed, and the S228P substitution is boxed and boldfaced.

In some embodiments, the anti-TGF-β antibody comprises one or more (e.g., all six) of the HCDR1-3 and the LCDR1-3 of fresolimumab. In other words, the antibody comprises one or more (e.g., all six) of the following HCDRs and LCDRs:

HCDR1
(SEQ ID NO: 4)
SNVIS
HCDR2
(SEQ ID NO: 5)
GVIPIVDIANYAQRFKG
HCDR3
(SEQ ID NO: 6)
TLGLVLDAMDY
LCDR1
(SEQ ID NO: 7)
RASQSLGSSYLA
LCDR2
(SEQ ID NO: 8)
GASSRAP
LCDR3
(SEQ ID NO: 9)
QQYADSPIT

In some embodiments, the anti-TGF-β antibody comprises the V_{H} and/or V_{L} of fresolimumab or Ab1. In other words, the antibody comprises one or both of the following sequences:

V_{H}:
(SEQ ID NO: 10)
QVQLVQSGAE VKKPGSSVKV SCKASGYTFS SNVISWVRQA
PGQGLEWMGG VIPIVDIANY AQRFKGRVTI TADESTSTTY
MELSSLRSED TAVYYCASTL GLVLDAMDYW GQGTLVTVSS
V_{L}:
(SEQ ID NO: 11)
ETVLTQSPGT LSLSPGERAT LSCRASQSLG SSYLAWYQQK
PGQAPRLLIY GASSRAPGIP DRFSGSGSGT DFTLTISRLE
PEDFAVYYCQ QYADSPITFG QGTRLEIK

In some embodiments, the anti-TGF-β antibody is of a human IgG isotype, such as human IgG₄ isotype. In certain embodiments, the human IgG₄ constant region comprises the following amino acid sequence:

(SEQ ID NO: 12)

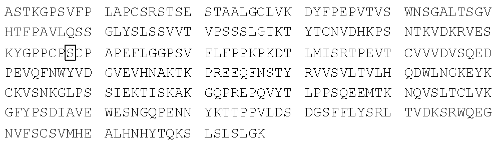

In further embodiments, the human IgG₄ constant region has a mutation at position 228 (Eu numbering). In some embodiments (e.g., Ab1), the mutation is a serine-to-proline mutation (S228P). In the above sequence, the S228 serine is boxed.

In some embodiments, the anti-TGF-β antibody (e.g., Ab1 and fresolimumab) comprises a human κ light chain constant region (C_{κ}). In certain embodiments, the human C_{κ} comprises the amino acid sequence:

(SEQ ID NO: 13)
RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

In some embodiments, an antigen-binding fragment of a full anti-TGF-β antibody may also be used. The term “antigen-binding fragment” or a similar term refers to the portion of an antibody that comprises the amino acid residues that interact with an antigen and confer on the binding agent its specificity and affinity for the antigen. Non-limiting examples of antigen-binding fragments include: Fab fragments, F(ab′)₂ fragments, Fd fragments, Fv fragments, single chain Fv (scFv), dAb fragments, and minimal recognition units consisting of the amino acid residues that mimic the hypervariable domain of the antibody.

In some embodiments, the antibody or antigen-binding fragment herein is connected to the bone-targeting moiety. In further embodiments, the bone-targeting moiety is a poly-arginine (poly-D) peptides. As used herein, the term “poly-D peptide” refers to a peptide sequence having a plurality of aspartic acid or aspartate or “D” amino acids, such as about 2, 3, 4, 5, 6, 7, 8, 9, 10, 20, 30, or more aspartic acid amino acids (residues). For example, a poly-D peptide can include about 2 to about 30, or about 3 to about 15, or about 4 to about 12, or about 5 to about 10, or about 6 to about 8, or about 7 to about 9, or about 8 to about 10, or about 9 to about 11, or about 12 to about 14 aspartic acid residues. Poly-D peptides may include only aspartate residues, or may include one or more other amino acids or similar compounds. As used herein, the term “D10” refers to a contiguous sequence of ten aspartic acid amino acids, as seen in SEQ ID NO:14. In some embodiments, an antibody or antibody fragment of the invention may include 1, 2, 3, 4, 5, 6, 7, 8, 9, 10, 11, 12, or more than 12 poly-D peptides.

The poly-D peptide can be connected to the anti-TGF-β antibody or antigen-binding fragment by fusion via recombinant technology, such that the poly-D is connected to the antibody or fragment through a peptidyl bond (i.e., the antibody or fragment is a fusion protein). For example, a poly-D peptide can be fused to the N- or C-terminus, or both, of the heavy chain, and/or the N- or C-terminus, or both, of the light chain. The poly-D peptide also can be connected to the anti-TGF-β antibody or antigen-binding fragment by chemical conjugation, e.g., by chemical reaction with a cysteine or lysine residue on the antibody or antibody-binding fragment with or without a linker moiety (e.g., a maleimide function group and a polyethylene glycol (PEG)). See, e.g., WO 2018/136698.

In certain embodiments, the antibody is fresolimumab fused to a D10 peptide at the N-terminus, C-terminus, or both termini, of the heavy chain. In some embodiments, the antibody is fresolimumab fused to a D10 peptide at the C-terminus of the light chain. In particular embodiments, the antibody is fresolimumab fused to a D10 peptide at both termini of the heavy chain and at the C-terminus of the light chain.

In certain embodiments, the antibody is Ab1 fused to a D10 peptide at the N-terminus, C-terminus, or both termini, of the heavy chain. In some embodiments, the antibody is Ab1 fused to a D10 peptide at the C-terminus of the light chain. In particular embodiments, the antibody is Ab1 fused to a D10 peptide at both termini of the heavy chain and at the C-terminus of the light chain.

The anti-TGF-β antibody or antigen-binding fragment thereof of the present disclosure can be made by methods well established in the art. DNA sequences encoding the heavy and light chains of the antibodies can be inserted into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody light chain coding sequence and the antibody heavy chain coding sequence can be inserted into separate vectors, and may be operatively linked to the same or different expression control sequences (e.g., promoters). The expression vectors encoding the antibodies of the present disclosure are introduced to host cells for expression. The host cells are cultured under conditions suitable for expression of the antibody, which is then harvested and isolated. Host cells include mammalian, plant, bacterial or yeast host cell. Mammalian cell lines available as hosts for expression are well known in the art and include many immortalized cell lines available from the American Type Culture Collection (ATCC). These include, inter alia, Chinese hamster ovary (CHO) cells, NSO cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines may be selected based on their expression levels. Other cell lines that may be used are insect cell lines, such as Sf9 or Sf21 cells. Tissue culture media for the host cells may include, or be free of, animal-derived components (ADC), such as bovine serum albumin. In some embodiments, ADC-free culture media is preferred for human safety. Tissue culture can be performed using the fed-batch method, a continuous perfusion method, or any other method appropriate for the host cells and the desired yield.

### III. Pharmaceutical Compositions and Use

The methods described herein comprise administering a therapeutically effective amount of an anti-TGF-β antibody or antigen-binding fragment thereof to an OI patient. As used herein, the phrase “therapeutically effective amount” means a dose of antibody that binds to TGF-β that results in a detectable improvement in one or more symptoms associated with OI (e.g., type I, II, III, or IV OI; or mild, moderate, moderate-to-severe, or severe type OD or which causes a biological effect (e.g., a decrease in the level of a particular biomarker) that is correlated with the underlying pathologic mechanism(s) giving rise to the condition or symptom(s) of OI.

Improvement of OI can be manifested in decreased bone turnover, reduced rates of bone remodeling, and/or decreased osteocyte density. In some embodiments, improvement in OI is indicated by improvement of a bone parameter selected from the group consisting of bone mineral density (BMD), bone volume density (BV/TV), total bone surface (BS), bone surface density (BS/BV), trabecular number (Tb.N), trabecular thickness (Tb.Th), trabecular spacing (Tb.Sp), and total volume (Dens TV).

In certain embodiments, the improved bone parameter is lumbar spine areal BMD (LS aBMD), as determined by dual-energy X-ray absorptiometry. Compared to baseline level prior to treatment, the LS aBMD value may increases by at least 1%, e.g., by at least 1, 2, 3, 4, 5, 6, 7, 8, 9, 10, 15, 20, or more percent.

In some embodiments, BMD, bone mass, and/or bone strength are increased by about 5% to about 200% following treatment with a therapeutically effective amount of the anti-TGF-β antibody or fragment. In certain embodiments, BMD, bone mass, and/or bone strength are increased by about 2%, about 2.1%, about 2.2%, about 2.3%, about 2.4%, about 2.5%, about 2.6%, about 2.7%, about 2.8%, about 2.9%, about 3%, about 4%, about 5% to about 10%, 10% to about 15%, 15% to about 20%, 20% to about 25%, 25% to about 30%, 30% to about 35%, 35% to about 40%, 40% to about 45%, 45% to about 50%, 50% to about 55%, 55% to about 60%, 60% to about 65%, 65% to about 70%, 70% to about 75%, 75% to about 80%, 80% to about 85%, 85% to about 90%, 90% to about 95%, 95% to about 100%, 100% to about 105%, 105% to about 110%, 110% to about 115%, 115% to about 120%, 120% to about 125%, 125% to about 130%, 130% to about 135%, 135% to about 140%, 140% to about 145%, 145% to about 150%, 150% to about 155%, 155% to about 160%, 160% to about 165%, 165% to about 170%, 170% to about 175%, 175% to about 180%, 180% to about 185%, 185% to about 190%, 190% to about 195%, or 195% to about 200%, following treatment.

In some embodiments, the therapeutically effective amount may lead to decreased bone turnover, e.g., as indicated by a decrease in serum or urinary biomarker such as urinary hydroxyproline, urinary total pyridinoline (PYD), urinary free deoxypyridinoline (DPD), urinary collagen type-I cross-linked N-telopeptide (NTX), urinary or serum collagen type-I cross-linked C-terminal telopeptide (CTX), bone sialoprotein (BSP), osteopontin (OPN), and tartrate-resistant acid phosphatase 5b (TRAP). In certain embodiments, the decrease, as compared to baseline level (e.g., before treatment), is by about 5% to about 200% following treatment with an antibody that binds to TGF-β. For example, the decrease may be about 5% to about 10%, 10% to about 15%, 15% to about 20%, 20% to about 25%, 25% to about 30%, 30% to about 35%, 35% to about 40%, 40% to about 45%, 45% to about 50%, 50% to about 55%, 55% to about 60%, 60% to about 65%, 65% to about 70%, 70% to about 75%, 75% to about 80%, 80% to about 85%, 85% to about 90%, 90% to about 95%, 95% to about 100%, 100% to about 105%, 105% to about 110%, 110% to about 115%, 115% to about 120%, 120% to about 125%, 125% to about 130%, 130% to about 135%, 135% to about 140%, 140% to about 145%, 145% to about 150%, 150% to about 155%, 155% to about 160%, 160% to about 165%, 165% to about 170%, 170% to about 175%, 175% to about 180%, 180% to about 185%, 185% to about 190%, 190% to about 195%, or 195% to about 200%, following treatment.

In some embodiments, the therapeutically effective amount may lead to an increase in the level of serum or urine biomarker of bone deposition, such as total alkaline phosphatase, bone-specific alkaline phosphatase, osteocalcin (OCN), and type-I procollagen (C-terminal/N-terminal). In certain embodiments, the increase, as compared to the baseline level (e.g., prior to treatment), is by about 5% to about 200% following treatment. For example, the increase may be by about 5% to about 10%, 10% to about 15%, 15% to about 20%, 20% to about 25%, 25% to about 30%, 30% to about 35%, 35% to about 40%, 40% to about 45%, 45% to about 50%, 50% to about 55%, 55% to about 60%, 60% to about 65%, 65% to about 70%, 70% to about 75%, 75% to about 80%, 80% to about 85%, 85% to about 90%, 90% to about 95%, 95% to about 100%, 100% to about 105%, 105% to about 110%, 110% to about 115%, 115% to about 120%, 120% to about 125%, 125% to about 130%, 130% to about 135%, 135% to about 140%, 140% to about 145%, 145% to about 150%, 150% to about 155%, 155% to about 160%, 160% to about 165%, 165% to about 170%, 170% to about 175%, 175% to about 180%, 180% to about 185%, 185% to about 190%, 190% to about 195%, or 195% to about 200% , following treatment.

In some embodiments, the therapeutically effective amount promotes bone deposition. In some embodiments, the therapeutically effective amount improves the function of a non-skeletal organ affected by OI, such as hearing, vision, lung function, and kidney function.

In some embodiments, the treatment with the anti-TGF-β antibody may be repeated every month, every two months, every three months, every four months, every five months, every six months, every nine months, every 12 months, or every 18 months. In some embodiments, the therapeutically effective amount of Ab1 may be 1-10 mg/kg, e.g., 1, 2, 2.5, 3, 4, 5, 6, 7, 8, 9, or 10 mg/kg, optionally administered twice a year (biannually, optionally every six months or Q6M). In other embodiments, the therapeutically effective amount of Ab1 may be a 0.1-1 mg/kg, e.g., 0.35, 0.4, or 0.5 mg/kg, optionally administered Q3M. In some embodiments, the OI patient is treated with this amount of Ab1 by intravenous injection. The treatment may be repeated at an interval as deemed appropriate by a physician for the patient.

The patients may be adults (e.g., patients 18 years or older). The patients may be pediatric patients (patients who are younger than 18 years old, e.g., patients who are newborn to 6 years old, who are 6 to 12 years old, or who are 12 to 18 years old).

### IV. Combination Therapies

In some embodiments, the present anti-TGF-β antibody therapy may be combined with other OI treatment. Examples of additional therapeutic agents include, but are not limited to, bisphosphonates, calcitonin, teriparatide, and any other compound known to treat, prevent, or ameliorate OI. The additional therapeutic agent(s) can be administered concurrently or sequentially with the antibody that binds to TGF-β. Examples of bisphosphonates are etidronate, clodronate, tiludronate, pamidronate, neridronate, olpadronate, alendronate, ibandronate, zoledronate, and risedronate. In some embodiments, the additional therapeutic agent is a drug that stimulates bone formation such as parathyroid hormone analogs and calcitonin.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: A Multi-Model Approach for Evaluating Anti-TGF-β Antibodies for Treatment of Osteogenesis Imperfecta

This example describes a study that characterized the concentration response relationship of anti-TGF-β antibody Ab1 and its impact on bone mineral density (BMD) and bone strength in OI patients. The study utilized model-based approaches informed from pre-clinical and clinical pharmacokinetics (PK) and pharmacodynamics (PD) data. Specifically, nonclinical PK/PD modeling was conducted using 1D11, and clinical PK/PD modeling was conducted with data obtained from cancer and OI patients, treated with fresolimumab (GC1008), or Ab1 during first-in-human studies. 1D11 was used as a surrogate rodent and is a pan-neutralizing TGF-β murine monoclonal antibody which binds with high affinity and neutralizes the biological activity of all three isoforms of TGF-β. Fresolimumab (GC1008) is a human anti-TGF-β monoclonal antibody that neutralizes all isoforms of TGF-β. Ab1 (GC2008) is a second generation human anti-TGF-β with high sequence similarity to fresolimumab (GC1008), only differing by a single amino acid in the heavy chain (S228P; Eu numbering). 1D11, fresolimumab (GC1008) and Ab1 represent molecules with identical mode of action differing only by their PK properties.

### Methods

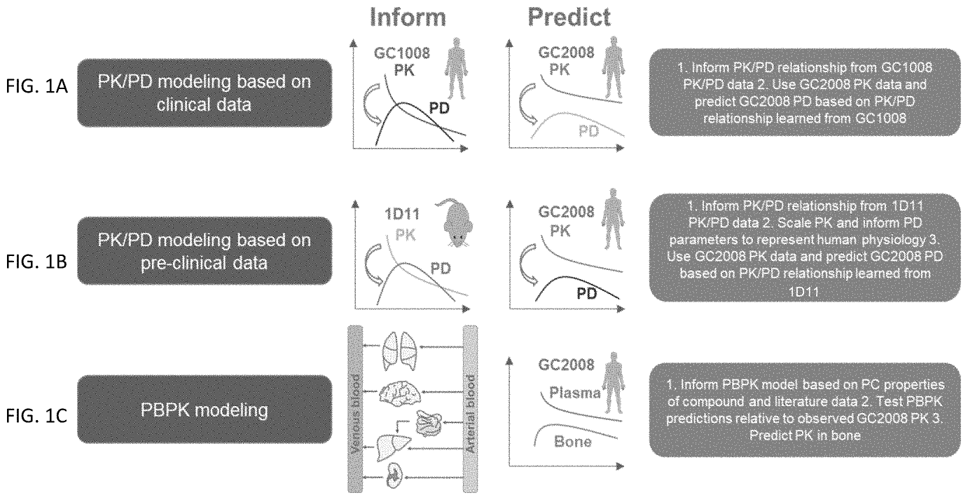

To understand the dose response relationship, three modeling approaches were developed: 1) a PK/PD approach based on PK and BMD clinical data from OI patients (FIG. 1A); 2) a PK/PD approach based on OI mouse pharmacology studies (FIG. 1B); 3) a physiological-based pharmacokinetic (PBPK) model approach to predict the dose that decreases OI TGF-β levels in bone to homeostatic levels (FIG. 1C).

In the first modeling approach, a PK/PD (BMD) relationship was established from GC1008; next, PK data from Ab1 (GC2008) was used along with the GC1008 PD-related parameters to provide a dose prediction. In the second modeling approach, a PK/PD (BV/TV, maxF) relationship was established from 1D11 data in mice. After scaling PK, PD parameters were informed based on human bone turnover rate, and the model was used to provide dose predictions. In the third modeling approach, a PBPK model was informed based on drug's physicochemical (PC) properties and human physiology. After verifying the validity of PBPK model's predictions by comparing with Ab1 PK data, PBPK model was used to evaluate bone PK and related target (TGF-β) profile. The multi-model approach is explained in further detail below.

### Exposure of Fresolimumab (GC1008) in Humans and PK Evaluation

PK of single-dose infusions of fresolimumab was evaluated during an open label, dose ranging first-in-human study conducted in patients with biopsy confirmed, treatment resistant, primary focal segmental glomerulosclerosis (FSGS). Sixteen patients received one of four single-dose levels of fresolimumab (0.3, 1, 2, 4 mg/kg) and were followed for 112 days, with rich sampling PK. The mean age of the patients was 37±12 years, mean FSGS duration was 3.0±2.1 years, half were male, 13 were White, and 3 were Black (Trachtman et al., Kidney Intern. (2011) 79 (11):1236-43). Serum PK of fresolimumab was best described by a two-compartment model with linear clearance (Trachtman et al., ibid). Patient weight was the only significant covariate identified as being predictive of pharmacokinetic variability. The half-life was estimated at 14 days, and mean dose normalized Cmax and exposures (AUC) did not change with dose. The PK model parameters are shown in Table 1.

TABLE 1
Input parameters for Fresolimumab (GC1008) PK/PD model

| | | |
|---|---|---|
| Parameters [Units] | Value | Source |

PK-related

| | | |
|---|---|---|
| V [L] | 3.28 | Estimated from PK |
| kel [1/hr] | 0.0039 | Estimated from PK |
| k12[1/hr] | 0.0059 | Estimated from PK |
| k21[1/hr] | 0.0064 | Estimated from PK |

Bone mineral density -PD-related

| | | |
|---|---|---|
| Bone turnover [hr] | 1080 | Indian J Endocrinol Metab. |
| | | (2016) 20(6): 846-52 |
| Bone Mineral density [g/cm²] | 0.75 | PD data |
| k_{in, 1} [1/hr] | 4.8135*10⁻⁴ | Baseline = kᵢₙ/kₒᵤₜ |
| k_{out, 1} [1/hr] | 6.418*10⁻⁴ | 1n(2)/Bone turnover/2 |
| E_{max, 1} | 0.9 | Estimated from PD |
| EC_{50, 1} [ng/ml] | 20000 | Estimated from PD |

### Fresolimumab Phase 1 Study in OI Patients

A phase 1 study with a single administration of fresolimumab was conducted in 8 adults with OI. The study involved a single infusion of fresolimumab (GC1008) (1 mg/ kg body weight and 4 mg/kg body weight; n=4 in each dose-cohort). Study's primary outcome was the safety of fresolimumab (GC1008) single administration whereas the effects of fresolimumab on bone remodeling biomarkers and lumbar spine areal bone mineral density (LS aBMD) were analyzed as secondary outcomes in a time frame of six months (Song et al., J Clin Invest. (2022) February 3:e152571. doi: 10.1172/JCI152571.

### Phase I Study of Ab 1 and PK Evaluation

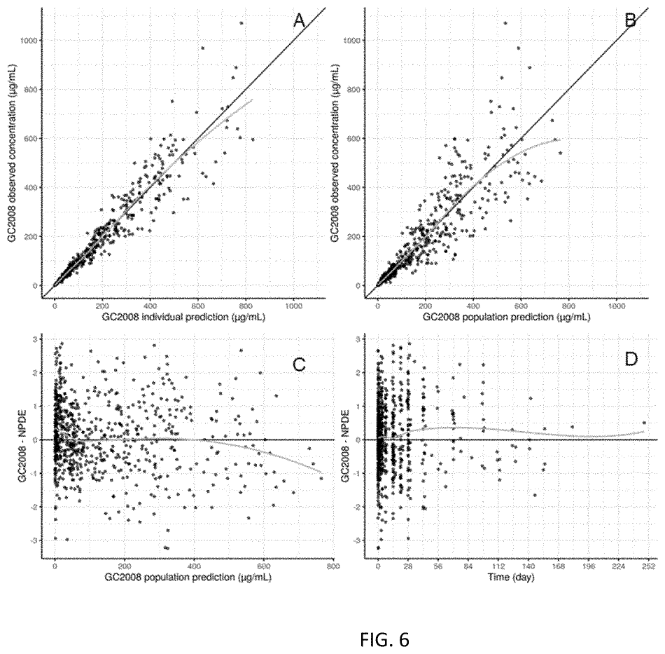

PK of Ab1 was evaluated during an open label, dose escalation, and expansion first-in-human study (NCT03192345) in cancer patients treated with Ab1 alone (Part A) or treated with Ab1 in combination with cemiplimab (Part B). A total of 52 patients received Ab1 30 minutes IV infusions, from 0.05 up to 15 mg/kg every 2 weeks (Q2W) or at 22.5 mg/kg every 3 weeks (Q3W). Blood samples for Ab1 measurement in serum were collected in all treated patients at start and end of drug infusion, 2.5, 4.5, 8.5 hours of day 1, at day 2, day 3, day 4, day 5, day 8 and day 15 (for Q2W) or day 22 (for Q3W) for cycle 1 (i.e., one cycle =one administration), at day 1 and day 8 of cycle 2, and day 1 in the subsequent cycles (Williamson et al., Developmental Therapeutics—Immunotherapy (2021) 39 (15_suppl):2510). The PK of Ab1 was similar to fresolimumab and was described by a two-compartment model with linear clearance (FIG. 6). The PK model parameters are shown in Table 2.

TABLE 2
Input parameters from for GC2008 PK/PD model

| | | |
|---|---|---|
| Parameters [Units] | Value | Source |

PK-related

| | | |
|---|---|---|
| V [L] | 3.49 | Estimated from PK |
| kel [1/hr] | 0.0017 | Estimated from PK |
| k12[1/hr] | 0.0074 | Estimated from PK |
| k21[1/hr] | 0.0112 | Estimated from PK |

PD-related

| | | |
|---|---|---|
| Bone turnover [hr] | 1080 | Jilka, R. L. J Gerontol A Biol |
| | | Sci Med Sci. (2013) 68(10): |
| | | p. 1209-17. |

Bone mineral density- PD related

| | | |
|---|---|---|
| Bone Mineral density | 0.75 | PD data |
| [g/cm²] | | |
| k_{in, 1} [1/hr] | 4.8135*10⁻⁴ | Baseline = kᵢₙ/kₒᵤₜ |
| k_{out, 1} [1/hr] | 6.418*10⁻⁴ | ln(2)/Bone turnover/2 |
| E_{max, 1} | 0.9 | Estimated from PD |
| EC_{50, 1} [ng/ml] | 20000 | Estimated from PD |

Bone Volume fraction-PD-related

| | | |
|---|---|---|
| Bone volume baseline [%] | 8.8 | OI patients |
| k_{in, 1} [%/hr] | 0.0056 | Baseline = kᵢₙ/kₒᵤₜ |
| k_{out, 1} [1/hr] | 0.000641 | ln(2)/Bone turnover/2 |
| E_{max, 1} | 2.2 | Estimated from PD |
| EC_{50, 1} [ug/ml] | 76.4 | Estimated from PD |

### 1D11 PK Study in OI Mouse Model

A single dose of 5 mg/kg 1D11 was administered intraperitoneally in the G610C OI mice (female/6 and male/6, eight weeks old) and blood was collected at 4, 48, 168, 360, 528, and 1032 hours post dose. All samples were processed for serum, placed on dry ice, and transferred to ≤−60° C. prior to analysis.

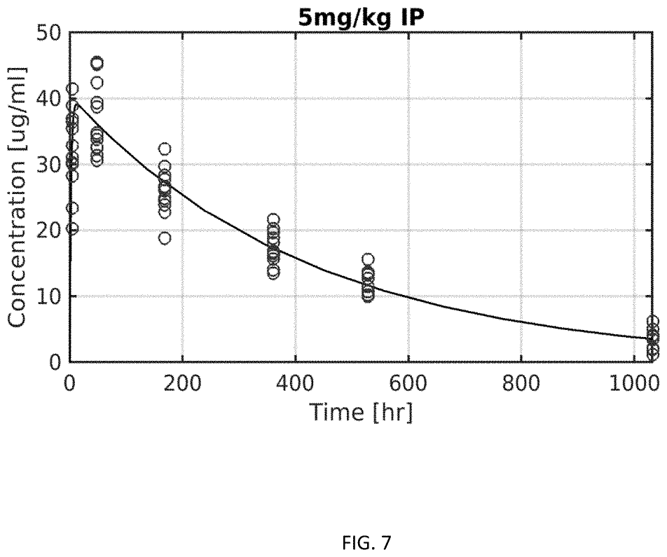

The circulating drug levels in serum were determined using an enzyme-linked immunosorbent assay (ELISA)-based bioanalytical method. Briefly, G610C mouse serum samples containing 1D11 were diluted in the buffer (PBS, 0.05% Tween-20, 0.05% Triton X-100, 0.01% BSA) at a 10,000-fold dilution for all samples except those from the last timepoint (1032 hours), which were diluted 1,000-fold. The 96-well plate was coated with TGF-β2, after incubated with mouse serum samples, 1D11 was captured using the detection antibody of goat anti-mouse horseradish peroxidase (HRP) conjugate (Sigma, A0168/095M4759V), followed by read the optical density at 450 nm and 570 nm in Spectramax® plus (Molecular Devices). The absorbance measured at 570 nm (background) was subtracted from the absorbance measured at 450 nm. A standard curve was generated and serum 1D11 concentrations were obtained. The low limit of detection of the assay was 1.0 μg/ml. The PK response of 1D11 is shown in FIG. 7. The PK parameters are shown in Table 3.

TABLE 3
Input parameters for preclinical mouse OI PK/PD model

| | | |
|---|---|---|
| Parameters [Units] | Value | Source |

PK-related

| | | |
|---|---|---|
| ka [1/hr] | 0.42 | Estimated from PK |
| V/F [ml/kg] | 124.06 | Estimated from PK |
| CL/F [ml/kg*hr] | 0.29 | Estimated from PK |

PD-related

| | | |
|---|---|---|
| Bone turnover [hr] | 336 | Jilka, et al., ibid |

Bone Volume fraction - PD

| | | |
|---|---|---|
| Bone volume baseline [%] | 10.42 | PD data (13C4-OI) |
| k_{in, 1} [%/hr] | 0.0028*10.42 | Baseline = kᵢₙ/kₒᵤₜ |
| k_{out, 1} [1/hr] | 0.0028 | ln(2)/Bone turnover/2 |
| E_{max, 1} | 2.2 | Estimated from PD |
| EC_{50, 1} [ug/ml] | 76.4 | Estimated from PD |

### In Vivo Pharmacology Study with 1D11 in OI Mice

Animals were provided with food (Auto KF 5 K 52; Lab Diet) and water ad libitum barrier- and gang-housed in pathogen-free, climate-controlled facilities with 12-hour light/dark cycles. The G610C OI (Stock no. 007248; Jackson Labs, hereafter referred to as OI mouse) mouse harbors a mutation in the Colla2 gene (Colla2tm1.1Mcbr), which results in a low bone mass and a brittle bone phenotype, thus representing a good preclinical model for autosomal dominant OI. A dose-dependent study was conducted as following: 1D11 was administered IP at 0.3, 1, or 5 mg/kg to male and female G610C OI mice at a dosing frequency of TIW over an 8-week period (n=4-8 males and 5-8 females per group). The pharmacodynamic effect of 1D11 across various doses was assessed. A dose-frequency study at a dose of 5 mg/kg of 1D11, administered IP either three times weekly, one time weekly, one time every 2 weeks, or one time every 4 weeks, for a total of 12 weeks was evaluated (n=5-8 for both uCT and biomechanics) (Greene, B., et al., JBMR Plus, (2021) 5 (9):e10530).

### Development of PK/PD Model for Fresolimumab and 1D11

The tiered approach followed in this work is shown in FIGS. 1A-1C. Initially, a PK/PD model was developed to evaluate the PK/BMD relationship of fresolimumab (GC1008) using the population PK model of fresolimumab performed in earlier studies (Trachtman et al., ibid) (FIG. 1A). The BMD dynamics were described by a type III indirect response model that simulates PK related increases in BMD through induction of the input rate of the effect compartment (Dayneka et al., J Pharmacokinet Biopharm. (1993) 21l (4):457-78). After informing the PD-related parameters with the available data (Song et al., ibid), fresolimumab PK was replaced by the 2-compartment PK model of GC2008 (Williamson et al., ibid) and based on the PK/PD relationship informed by fresolimumab, the model was used to provide predictions for the Ab1 dose/response (BMD) relationship. Population PK analysis of fresolimumab and Ab1 was performed using NONMEM and Monolix respectively (Bauer et al., CPT Pharmacometrics & Systems Pharmacology (2019) 8 (8):525-537). PK/PD modeling was performed in Matlab R2019a using ode45 solver for ordinary differential equations.

In the second modeling approach, a PK/PD relationship was established for 1D11 in mice (FIG. 1B). PD endpoints measured were bone volume fraction (bone volume/total volume−BV/TV), and maximum force to failure (maxF), both representing amelioration of bone physiology. 1D11 PK was described by a 1-compartment model ((FIG. 7), Table 3) and the dynamics of BV/TV and maxF by a type III indirect response model. To translate PK/PD relationship to humans, Ab1 pop-PK model was used, and the mouse bone turnover rate (−3 weeks) was replaced by the human bone turnover rate (−3 months) while the PD related parameters were kept constant. The model was then used to predict Ab1 dose/response (BV/TV) relationship. PK/PD modeling for 1D11 and its forward translation to humans was performed in Matlab R2019a using ode45 solver for ordinary differential equations.

### PBPK Model for Ab1

Lastly, a PBPK modeling approach was used to evaluate Ab1 PK in bone, and the corresponding TGF-β response in humans. Based on the physicochemical properties of Ab1, TGF-β levels in plasma and bone, and human physiology, a PBPK model was developed using the PK-Sim® software platform (Willmann et al., BIOSILICO (2003) 1 (4):121-1240). To validate PBPK predictions, Ab1 clinical PK data were compared with PBPK simulations for the according scenarios. After validation, the PBPK model was used to evaluate PBPK/TGF-β response in human plasma and bone tissue. The PK parameters are shown in Table 4.

TABLE 4
PBPK model input parameters for Ab1

| Parameters | Value | |
|---|---|---|
| [Units] | [Units] | Source |
| Baseline of | 230 | (Mancini et al., Transl Res. |
| TGF-β 1 in | [pmol/L] = 5750[pg/ml] | (2018) 192: 15-29; |
| plasma | | Grainger et al., Nat Med. |
| [pg/ml] | | (1995) 1(1): 74-9) |
| Baseline of | TGF-β 1: 0.23-0.87 | Pfeilschifter et al., J Bone Miner Res. |
| TGF-β in | [ng/mg] | (1998) 13(4): 716-30) |
| bone | TGF-β 2: 0.00929- | |
| (healthy and | 0.01448 [ng/mg] | |
| OI) | 3 times more in the | |
| | disease (both system | |
| | and bone)/MR's | |
| | model | |
| Half-life of | 1 | (Wakefield et al., J Clin Invest. |
| TGF-β in | | (1990) 86(6): 1976-84). |
| plasma [min] | | |
| kon/koff to | 3.17E+05/4.31E−04 | (Greco et al., Oncoimmunology |
| TGF-β 1 | | (2020) 9(1): 1811605). |
| [1/M*s]/[1/s] | | |
| kon/koff to | 2.51E+05/7.93E−04 | (Greco et al., ibid) |
| TGF-β 2 | | |
| [1/M*s]/[1/s] | | |
| kon/koff to | 2.04E+05/2.64E−04 | (Greco et al., ibid) |
| TGF-β 3 | | |
| [1/M*s]/[1/s] | | |
| Kd at pH 6.0 | 1400 | Unpublished data |
| [nM] | | |
| MW [kDa] | 25 | https://www.uniprot.org/uniprot/A0A024R0P8 |

### Results

### First Modeling Approach

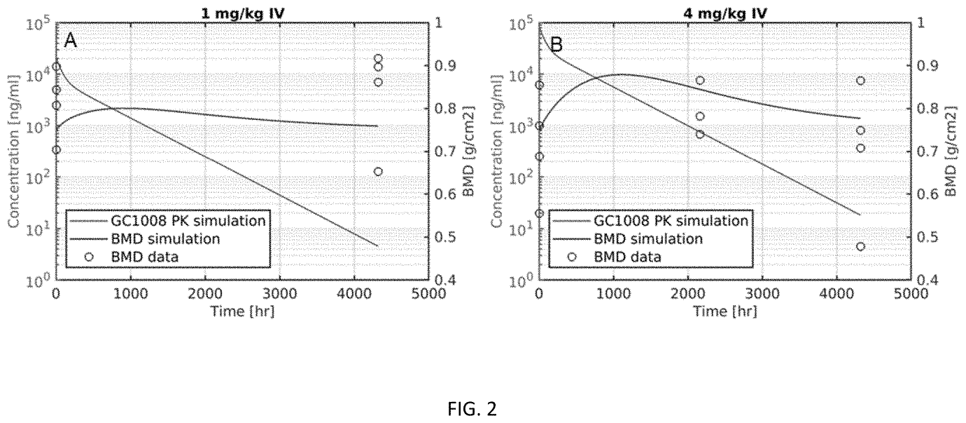

PK of fresolimumab in the serum of focal segmental glomerulosclerosis (FSGS) was evaluated in previous studies (Trachtman et al., ibid). PK/BMD response of fresolimumab in OI patients was explored by using a type III indirect response model for BMD (FIG. 2). In the first modeling approach, a PK/PD model was initially informed by the fresolimumab (GC1008) PK/BMD data. FIG. 2 shows the PK/BMD dynamics, along with the respective BMD data for single dose of 1 and 4 mg/kg fresolimumab (GC1008), in OI patients (Song et al., ibid). Parameters of the PD model were fit to the BMD data after administration of 1 (FIG. 2, Graph A) and 4 mg/kg (FIG. 2, Graph B) fresolimumab (GC1008). For BMD, administration of 1mg/kg had minimal effect on the dynamics as shown in FIG. 2, Graph A. The simulations suggest a more profound increase in the initial period after a 4 mg/kg IV dose. In both dose groups, number of patients was low and their BMD values maintained significant variability. The model was able to explain available data successfully.

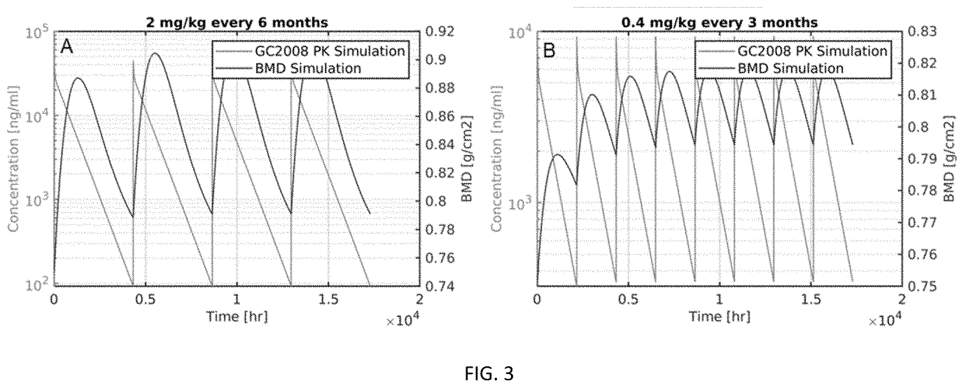

In the second step of this initial modeling approach, the PK part of the PK/PD model was further informed from the Ab1 PK data whereas the PD parameters were kept constant. PK of Ab1 was further incorporated in the model based on prior pop-PK analysis. BMD-related parameters were kept constant to those of fresolimumab. FIG. 3 shows the PK/BMD simulated response of Ab1 when administered IV as 2 mg/kg every six months (FIG. 3, Graph A), and 0.4 mg/kg administered IV every three months (FIG. 3, Graph B). The doses shown in FIG. 3 are the doses resulting in a 5% increase in the BMD. Hence, PK/BMD model of Ab1 predict a 2 mg/kg bi-annual administration (FIG. 3, Graph A) or 0.4 mg/kg administration every 3 months to increase BMD by 5% (FIG. 3, Graph B).

Fresolimumab PK behavior in FSGS was similar in two other disease populations namely idiopathic pulmonary fibrosis and advanced malignancy (Morris et al., PLoS One (2014) 9(3):e90353). The underlying hypothesis in this work is that fresolimumab is expected to have a similar pharmacokinetic profile in OI patients, and therefore the PK parameters derived from the pop-PK analysis of fresolimumab in cancer patients can be used to describe the PK of fresolimumab (GC1008) in OI patients. As shown in the examples herein, for modeling the PD dynamics after fresolimumab (GC1008) administration, a type III PD model was used. This model represents drug response that accrues from stimulation of the factors controlling the production of the response variable, which in this case is BMD (Dayneka et al., ibid). Although a type II model that represents drug response accruing form inhibition of the dissipation of the response could also be used to model the BMD data, in some embodiments, a type III model is preferred based on the underlying physiology where anti-TGF-β treatment ultimately blocks the mechanism inducing BMD (Bonewald et al., Clin Orthop Relat Res. (1990) (250):261-76). Due to the low number of BMD data (4 subjects), their sparsity, and their high variability (FIG. 2, Graphs A and B), the Eₘₐₓ/EC₅₀ parameters of the PD model were optimized according to the average BMD value for each time point, whereas kin/kout were set based on bone turnover, and BMD baseline in humans. The model predicts minimal effect on BMD after a single dose of 1 mg/kg Fresolimumab (GC1008), whereas administration of 4 mg/kg induces a stronger effect with a more pronounced increase of BMD the first hundred days. As shown in the examples herein, the BMD response after Ab1 administration was assumed to follow the same dynamics (same PD-model, and related parameters) as the ones informed from fitting PK/BMD of fresolimumab. Therefore, and in some embodiments, using the population-PK derived parameters of Ab1, and the BMD-related parameters identified in the first step of this modeling approach, our model provides predictions on PK/PD response of Ab1 (FIG. 3, Graphs A and B).

To date, there are several studies investigating the effects of various treatments to BMD in OI patients. In a clinical study involved twenty-three men and twenty-three premenopausal women with OI, Adami et al. tested the effect of neridronate, an amino-bisphosphonate, when administered every three months (Adami et al., ibid). Within the first twelve months of treatment, spine and hip bone mineral density rose by 3% and 4.3% respectively, and during the second year of follow up additional 3.91% and 1.49% increases were observed. The magnitude of these changes was considered clinically relevant based on the relationship between BMD changes and fracture risk reduction (Hochberg et al., J Clin Endocrinol Metab. (2002) 87 (4):1586-92). In the clinical trial of Orwoll et al. seventy-nine adults with OI were randomized at a 1:1 ratio to receive subcutaneous 20 μg/day teriparatide or placebo. Compared to the placebo group, the treatment group showed a 6.1% increase in lumbar spine (LS) areal BMD (aBMD) vs 2.8%, and total hip aBMD 2.6% vs −2.4%. Furthermore, vertebral BMD (vBMD) and strength improved with the treatment but declined with placebo. Overall, the results indicated that adults with OI displayed an increased hip and spine aBMD, vBMD and estimated strength. In the retrospective analysis of (Kuhn et al., J Musculoskelet Neuronal Interact. (2014) 14 (4):445-53) the effect of the new physiotherapy approach including side alternating whole body vibration on motor function was analyzed in 53 children with OI. After 12 months, the children showed a significant increase of motor function and walking distance that was accompanied with an increase of the aBMD from 0.4357 to 0.48 (˜10%) and of the BMD of total body without head from 0.5382 to 0.5529 (˜3%). Finally, in a later study Kuhn et al. showed that denosumab, a RANK ligand antibody inhibiting osteoclast maturation, led to a 19% increase in the lumbar aBMD in ten children with OI (Kuhn et al., ibid). In conclusion, current data indicate that significant amelioration in clinical outcome is expected for 5% increase in BMD. Based on the model-based prediction of Ab1 PK/PD, a 5% BMD increase is achieved when 0.4 mg/kg Ab1 are administered once every three months (FIG. 3, Graph A), or 2 mg/kg every six months (FIG. 3, Graph B).

### Second Modeling Approach

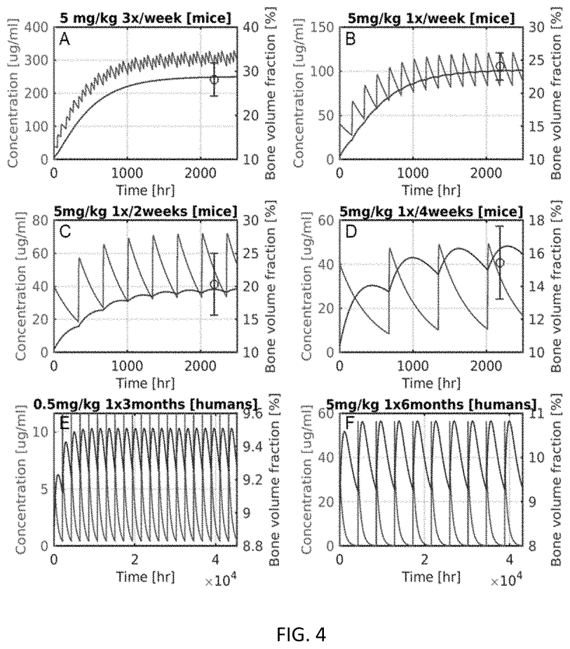

In the second modeling approach, PK of 1D11 was evaluated in mice (FIG. 7). Mice 1D11 PK was modeled using 1CM. A PK/PD model was further developed based on the change in bone volume. Bone volume fraction changes were further evaluated by a type III IDR model (Benjamin et al., JMBR Plus 5.9 (2021):e10530). To use the pre-clinical model for human predictions, bone turnover rate and bone volume fraction baseline were adjusted with the human values (FIG. 4). The PD parameters were fitted to the mouse PD. FIG. 4 shows the PK/PD response after intravenous administration of 5 mg/kg 1D11 after various regimens. Comparing the simulated (solid lines) with the experimental observations (symbols), the model was able to describe the observed data satisfactorily. The model further predicts that more frequent administration of the same dose leads to a faster time to reach steady state of the PD response. To predict PD response in humans, PK of Ab1 was used, and the baseline along with turnover rate parameters of the PD model were changed to represent human values of bone volume fraction and bone turnover accordingly. FIG. 4, Graph E and Graph F depict model-based PK/PD predictions for 0.5 mg/kg IV administration once per 3 months, and 2.5 mg/kg IV administration every 6 months in humans, respectively. These doses result in a 5% increase on bone volume fraction. PK/BV model of Ab1 predicts a 2.5 mg/kg administration bi-annually (FIG. 4, Graph F) or 0.5 mg/kg administration every 3 months to increase BV by 5% (FIG. 4, Graph E).

In the second modeling approach (FIG. 1B), available pre-clinical data of 1D11 are taken into consideration. The PK of 1D11 was described using a one-compartment model with linear clearance. Although a two-compartment model explained the PK data equally well, the confidence interval of the parameters of the second compartment were low and as such the 1 compartment model was chosen. In accordance with the first modeling approach described earlier, a type-III indirect response model was used to describe the bone volume fraction changes in mice (FIG. 4, Graphs A-D). kᵢₙ/kₒᵤₜ were set according to bone turnover, and bone volume fraction in mice, and the Eₘₐₓ/EC₅₀ were optimized based on the available bone volume fraction data. As seen in FIG. 4, 1D 11 mice PK/PD model was able to describe the available data satisfactorily. Of note, bone volume fraction measurements were available for one time point limiting the predicting capacity of the model especially for the intermediate time points. To translate the mice 1D11 PK/PD model to GC2008 PK/PD in humans, three steps were taken. Initially, the 1D11 PK model was replaced by the GC2008 PK model evaluated previously. Furthermore, the mice bone volume fraction baseline was replaced by the literature-based value of bone volume fraction in OI patients (Glorieux et al., J Bone Miner Res. (2000) 15 (9):1650-8; and Glorieux et al., J Bone Miner Res. (2002) 17 (1):30-8), and the bone turnover of mice which is nearly three weeks was replaced by the nearly three months value of the human bone turnover (Jilka, ibid). Based on these changes, the model was used to evaluate the PK/PD response of GC2008 as shown in (FIG. 4, Graph E and Graph F). To achieve a 5% increase in bone volume fraction, PK/PD model predicts administration of 0.5 mg/kg every three month, or 2.5 mg/kg bi-annually.

### Third (Last) Modeling Approach

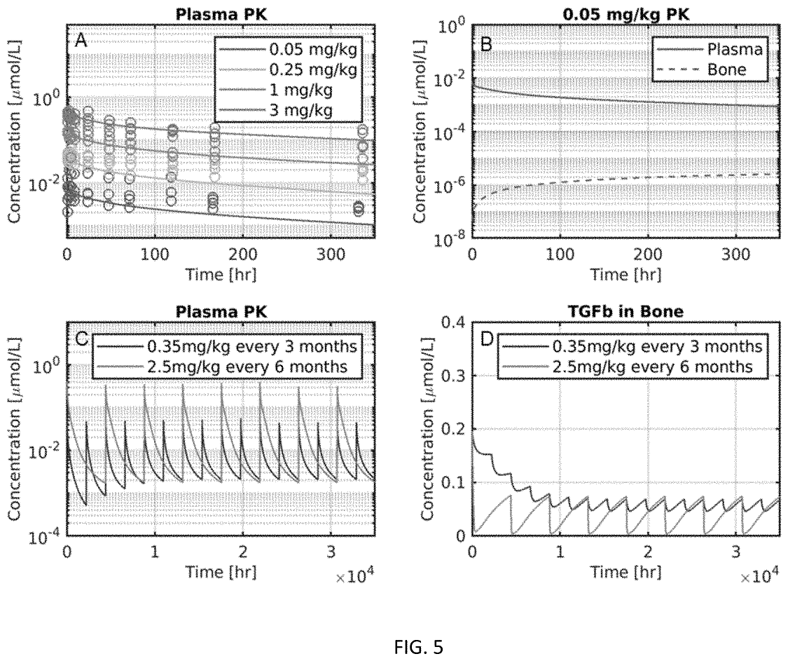

In the last modeling approach, a PBPK model was developed for Ab1, and used to predict the dose needed to reduce TGF-β in bone to its physiological level. The PBPK model developed incorporates physicochemical properties of Ab1 along with information regarding TGF-β expression in plasma and bones of healthy and OI patients. PBPK model-based predictions of Ab1 PK for multiple doses were in close accordance with the available data. FIG. 5 shows validation of the PBPK model and its forward predictions. FIG. 5, Graph A illustrates the response of the PBPK model for different doses of Ab1. Solid lines depict model-based predictions and open circles the individual clinical PK data for the different doses. Comparison between simulations and the PK data indicates that the PBPK model predicts drug exposure in humans well, for scenarios that were not used to train the model. FIG. 5, Graph B depicts the distribution of Ab1 in plasma (solid line) and bone (dotted line), for 0.05 mg/kg IV administration of Ab1. The PBPK model predicts that concentration in bone is nearly 5% of that in plasma. To simulate an OI scenario, TGF-β expression was increased to represent the three times higher concentration of TGF-β in the OI patients. FIG. 5, Graph C further depicts PBPK model-based PK prediction of OI patients, where 0.35 mg/kg and 2.5 mg/kg IV administration of Ab1 was administered every three and six months respectively. These doses were found to decrease TGF-β levels to their physiological values for the according dosing schemes. FIG. 5, Graph D further depicts the corresponding TGF-β target levels after 0.35 mg/kg and 2.5 mg/kg IV administration of Ab1 every three and six months. The PBPK model predicts a dose of 0.35 mg/kg every 3 months and 2.5 mg/kg every 6 months in order decrease TGF-β levels to their homeostatic value (FIG. 5).

In the last modeling effort, a PBPK approach was implemented to predict the effect of Ab1 on the levels of TGF-β in bone. It is well known that PBPK models have an optimal mathematical framework based on which distribution of drug in the different tissues is predicted depending on physiological-based mass balances and transport phenomena (Jones and Rowland-Yeo., CPT Pharmacometrics Syst Pharmacol. (2013) 2:e63). The input to PBPK can be generally divided to drug-specific, and organism-specific parameters. Drug-specific parameters are related to the physicochemical properties of the compound such as molecular weight, affinity to FcRn, affinity to the target of interest and others. Organism-specific parameters are related to physiological characteristics of the body such as tissue volumes, and tissue blood flows, which are mostly based on literature and generally incorporated in the model platform used. Given their significance in model-based drug development, there are several commercial platforms that integrate physiologically based methodologies such as Simcyp (certara website), GastroPlus (simulations-plus website), SimBiology (mathworks website), and PK-Sim (open-systems-pharmacology website). The PK-Sim platform was used due its relative ease of incorporating target binding in the tissue of interest. The distribution model that was used to describe the kinetics of Ab1 was based on the two-pore formalism and was previously described (Niederalt et al., J Pharmacokinet Pharmacodyn. (2018) 45 (2):235-57). The input parameters needed were the baseline concentration of TGF-β in plasma and bone, the binding affinities to TGF-β and FcRn, and the molecular weight of Ab1. To evaluate the predicting capacity of the model, simulations were compared with the available PK data of Ab1 (FIG. 5, Graph A). Model-based predictions were able to describe the available data well and increased the confidence of the model predictions, especially since the model has not been previously trained on the Ab1 PK data. Although there was not available bone Ab1 PK to compare the predicted distribution, our PBPK predictions are well aligned with the literature indicating an average of 7% distribution of large molecules to bone (FIG. 5, Graph B) (Shah and Betts, MAbs. (2013) 5 (2):297-305). After establishing confidence for PBPK model predictions, a scenario of increased TGF-β concentration was implemented. Based on the available literature evidence, OI patients showed nearly 3 times higher concentration of TGF-β in plasma and bone relative to healthy individuals (Grafe et al., Nat Med. (2014) 20 (6):670-5; Gebken et al., Pathobiology (2000) 68(3):106-12; and Pfeilschifter et al., ibid). Based on this piece of evidence, to simulate an OI scenario, the TGF-β expression in plasma and bone was increased. Hence, we sought to evaluate the dose of Ab1 that reduces the OI related free TGF-β levels back to their healthy value. The underlying assumption is that OI related changes in body physiology (i.e., reduced bone volume) do not impact Ab1 PK and can remain constant. Based on the PBPK analysis, administration of 0.35 mg/kg every 3 months or 2.5 mg/kg every 6 months (FIG. 5, Graph C) will lead the free TGF-β levels in bone back to their physiological values (FIG. 5, Graph D). Interestingly, PBPK analysis indicated that 2.5 mg/kg administration bi-annually eliminates almost the total amount of free TGF-β in bone when Ab1 concentration reaches its peak. This further unveils a constraint for possible dosing designs where administration should be optimized to account for this implication.

In summary, a multi-model approach was implemented to evaluate the concentration response relationship of an anti-TGF-β antibody and BMD and bone strength, and the TGF-β dynamics in bone of OI patients. The three modeling approaches provided a similar dose projection for clinically relevant PD effects. The three modeling approaches implemented in this work provided a similar dose estimate for clinically relevant PD effects. In particular, the first approach using fresolimumab, Ab1 clinical PK/PD data predicted a 0.4 mg/kg administration every 3 months or 2 mg/kg bi-annually to increase the BMD 5%. The second approach which further used pre-clinical data of 1D11 predicted a 0.5 mg/kg administration every 3 months and 2.5 mg/kg administration bi-annually to increase bone volume fraction 5%. Finally, PBPK modeling predicts a 0.35 mg/kg administration every 3 months or 2.5 mg/kg administration bi-annually to decrease OI-related TGF-β levels back to their physiological values. Correspondence of the three approaches, increased the confidence for the translation of the PK/PD relationship of Ab1 and provided a robust model-based evaluation for predicting clinical efficacy.

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

### Sequences

The table below shows the amino acid sequences referred to in the present disclosure.

| SEQ ID NO | Description |
|---|---|
| 1 | Fresolimumab heavy chain |
| 2 | Fresolimumab or Ab1 light chain |
| 3 | Ab1 heavy chain |
| 4 | Fresolimumab or Ab1 HCDR1 |
| 5 | Fresolimumab or Ab1 HCDR2 |
| 6 | Fresolimumab or Ab1 HCDR3 |
| 7 | Fresolimumab or Ab1 LCDR1 |
| 8 | Fresolimumab or Ab1 LCDR2 |
| 9 | Fresolimumab or Ab1 LCDR3 |
| 10 | Fresolimumab or Ab1 V_{H} |
| 11 | Fresolimumab or Ab1 V_{L} |
| 12 | Human IgG₄ constant region |
| 13 | Human κ light chain constant region |
| 14 | D10 amino acid sequence |

1. A method for treating osteogenesis imperfecta (OI) in a human subject in need thereof, comprising administering to the subject a therapeutically effective amount of an anti-TGF-β antibody,
wherein the antibody comprises heavy chain complementarity-determining regions (CDRs) 1-3 comprising SEQ ID NOs:4-6, respectively, light chain CDR1-3 comprising SEQ ID NOs:7-9, respectively, wherein the antibody comprises a human IgG4 constant region having a proline at position 228 (Eu numbering), and
wherein the therapeutic effective amount is
1 to 8 mg/kg, optionally 2, 2.5, or 5 mg/kg, administered bi-annually, or 0.1 to 1 mg/kg, optionally 0.35, 0.4, or 0.5 mg/kg, administered every 3 months (Q3M).

2. The method of claim 1, wherein the antibody comprises a heavy chain variable domain comprising SEQ ID NO:10 and a light chain variable domain comprising SEQ ID NO:11.

3. The method of claim 1, wherein the antibody comprises a human IgG4 constant region and/or a human κ light chain constant region.

4. The method of claim 3, wherein the antibody comprises a heavy chain comprising SEQ ID NO:3 and a light chain comprising SEQ ID NO:2.

5. The method of claim 1, wherein the antibody comprises a bone-targeting moiety, optionally wherein the bone-targeting moiety is a poly-arginine peptide.

6. The method of claim 5, wherein the antibody comprises one or more poly-arginine peptides.

7. The method of claim 6, wherein the antibody is fused to a poly-arginine peptide at the N-terminus, or the C-terminus, or both termini, of the heavy chain, and/or at the C-terminus of the light chain, of the antibody.

8. The method of claim 5, wherein the poly-arginine peptide is D10 (SEQ ID NO:14).

9. The method of claim 1, wherein the OI is moderate-to-severe OI or type IV OI.

10. The method of claim 1, wherein the OI is type I, II, or III OI.

11. The method of claim 1, wherein the human subject is an adult patient (≥18 years of age), or a pediatric patient (<18 years of age).

12. The method of claim 1, wherein the human subject has a mutation in a COL1A1 or COL1A2 gene, optionally wherein the mutation is a glycine substitution mutation in the COL1A1 or COL1A2 gene or a valine deletion in the COL1A2 gene.

13. The method of claim 1, wherein the administration improves a bone parameter selected from the group consisting of bone mineral density (BMD), bone volume density (BV/TV), total bone surface (BS), bone surface density (BS/BV), trabecular number (Tb.N), trabecular thickness (Tb.Th), trabecular spacing (Tb.Sp), and total volume (Dens TV).

14. The method of claim 1, wherein the administration decreases bone turnover and/or osteocyte density, optionally wherein the decreased bone turnover is indicated by a decrease in serum CTX or an increase in serum osteocalcin (OCN).

15. The method of claim 1, wherein the antibody is administered at 2 mg/kg bi-annually or at 0.4 mg/kg Q3M, optionally wherein the administration leads to an increase of BMD in the subject by about 5%.

16. The method of claim 1, wherein the antibody is administered at 5 mg/kg bi-annually or at 0.5 mg/kg Q3M, optionally wherein the administration leads to an increase of BV in the subject by about 5%.

17. The method of claim 1, wherein the antibody is administered at 2.5 mg/kg bi-annually or at 0.35 mg/kg Q3M, optionally wherein the administration leads to a decrease of the TGF-β level in the subject to the homeostatic value.

18. The method of claim 1, wherein the antibody is administered by intravenous infusion.

19. The method of claim 1, further comprising administering to the subject a bisphosphonate, parathyroid hormone, calcitonin, teriparatide, or an anti-sclerostin agent.

20. The method of claim 19, wherein the bisphosphonate is selected from alendronate, pamidronate, zoledronate, and risedronate.

21. An article of manufacture or kit, comprising an anti-TGF-β antibody for use in treating osteogenesis imperfecta in the method of claim 1.


SEQUENCE LISTING (14 sequences)

SEQ ID NO: 1 - protein, 447 aa, synthetic construct
  REGION 1..447: note = Synthetic: Fresolimumab heavy chain
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 215 aa, synthetic construct
  REGION 1..215: note = Synthetic: Fresolimumab or Ab1 light chain
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
  REGION 1..447: note = Synthetic: Ab1 heavy chain
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 5 aa, synthetic construct
  REGION 1..5: note = Synthetic: Fresolimumab or Ab1 HCDR1
SNVIS

SEQ ID NO: 5 - protein, 17 aa, synthetic construct
  REGION 1..17: note = Synthetic: Fresolimumab or Ab1 HCDR2
GVIPIVDIANYAQRFKG

SEQ ID NO: 6 - protein, 11 aa, synthetic construct
  REGION 1..11: note = Synthetic: Fresolimumab or Ab1 HCDR3
TLGLVLDAMDY

SEQ ID NO: 7 - protein, 12 aa, synthetic construct
  REGION 1..12: note = Synthetic: Fresolimumab or Ab1 LCDR1
RASQSLGSSYLA

SEQ ID NO: 8 - protein, 7 aa, synthetic construct
  REGION 1..7: note = Synthetic: Fresolimumab or Ab1 LCDR2
GASSRAP

SEQ ID NO: 9 - protein, 9 aa, synthetic construct
  REGION 1..9: note = Synthetic: Fresolimumab or Ab1 LCDR3
QQYADSPIT

SEQ ID NO: 10 - protein, 120 aa, synthetic construct
  REGION 1..120: note = Synthetic: Fresolimumab or Ab1 VH
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 11 - protein, 108 aa, synthetic construct
  REGION 1..108: note = Synthetic: Fresolimumab or Ab1 VL
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 12 - protein, 327 aa, Homo sapiens
  REGION 1..327: note = misc_feature - Human IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 107 aa, Homo sapiens [sequence omitted]

SEQ ID NO: 14 - protein, 10 aa, synthetic construct
  REGION 1..10: note = Synthetic: D10 amino acid sequence
DDDDDDDDDD